(19) United States
(12) Patent Application Publication
(10) Pub. No.: US 2022/0192779 A1
(43) Pub. Date: Jun. 23, 2022

(54) Upper Body Shield

(71) Applicant: Angela Moore, Madison, AL (US)

(72) Inventor: Angela Moore, Madison, AL (US)

(21) Appl. No.: 17/130,267

(22) Filed: Dec. 22, 2020

(51) Int. Cl. A61B 90/40; A61B 46/20

(52) U.S. Cl. CPC A61B 90/40; A61B 2046/201; A61B 46/20

(57) ABSTRACT

An upper body shield that has a headrest to balance and support the upper body shield. The upper body shield further has at least one curved frame arm configured to attach to the headrest, a top cover that couples to the at least one curved frame arm configured to shield a patient's head and neck, and a drape configured to couple to the at least one frame arm and configured to shield sides of the patient.

### BACKGROUND

Oftentimes, medical professionals are required to perform procedures on patients, e.g., intubation. Intubation is a process of the process of insertion of a tube, also called an endotracheal tube (ET), through the mouth and then into the airway of a patient. Intubation is done so that the patient can be placed on a ventilator. The ventilator assists with breathing during anesthesia, sedation, or severe illness. The ET is connected to the ventilator, which pushes air into the lungs to deliver a breath to the patient.

While the patient is intubated, the medical professionals may be required to perform medical procedures to patients at or around the head, neck, or chest of the patient. Exemplary types of procedures include, but are not limited to, intubating/extubating, monitored anesthesia care, ventilation, or prepping the face for a surgical procedure.

While performing procedures on intubated patients or other patients who have not necessarily been intubated, it is best if the medical professional who is performing a procedure not be exposed to the patient's respiratory spray. For example, the patient may have contracted a communicable disease, such as COVID-19.

Further, it is oftentimes best if the patient is not exposed to the medical professional. For example, the medical professional may have contracted a communicable disease and be presently asymptomatic.

Thus, it would be best if the patient is shielded from the medical professional and the medical professional is shielded from the patient while procedures are performed on the patient. However, it is beneficial if while shielding the patient, the medical professional is still able to perform the procedures, as described above, on the patient.

### BRIEF DESCRIPTION OF THE DRAWINGS

The present disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1 is a perspective view of an upper body shield in accordance with an embodiment of the present disclosure depicting a transparent top cover attached to frame arm.

FIG. 2 is a perspective view of the upper body shield of FIG. 1 further depicting a transparent drape coupled to the transparent top cover and/or the frame arms.

FIG. 3 is a side view of the upper body shield of FIG. 1 depicting the transparent top cover coupled to the frame arms.

FIG. 4 is a side view of the upper body shield of FIG. 2 showing the transparent drape coupled to the transparent top cover and/or the frame arms.

FIG. 5 is a back end view of the upper body shield of FIG. 1 depicting the transparent top cover attached to the frame arms.

FIG. 6 is a back end view of the upper body shield of FIG. 2 depicting the transparent drape coupled to the transparent top cover and/or the frame arms.

### DETAILED DESCRIPTION

The present disclosure describes and upper body shield in accordance with an embodiment of the present disclosure. The exemplary upper body shield comprises a headrest. Further, the upper body shield comprises at least two frame arms, each coupled toward opposing sides on a back side of the headrest.

The upper body shield further comprises a transparent top cover. The transparent top cover couples to the frame arms and provides cover to the patient/medical professional of the head of a patient.

The upper body shield further comprises a transparent drape. The transparent drape couples to the frame arms and/or the upper shield. The drape provides shielding of the patient on the back side, each lateral side, and a front side. In this regard, the drape is attached to the frame arms and/or the upper shield and falls around the patient, covering the patient's chest, neck, and head. Taken together, the upper shield and the drape limit exposure of the patient from the medical professional and limit exposure of the medical professional from the patient while the medical professional is performing procedures, e.g., intubation, etc.

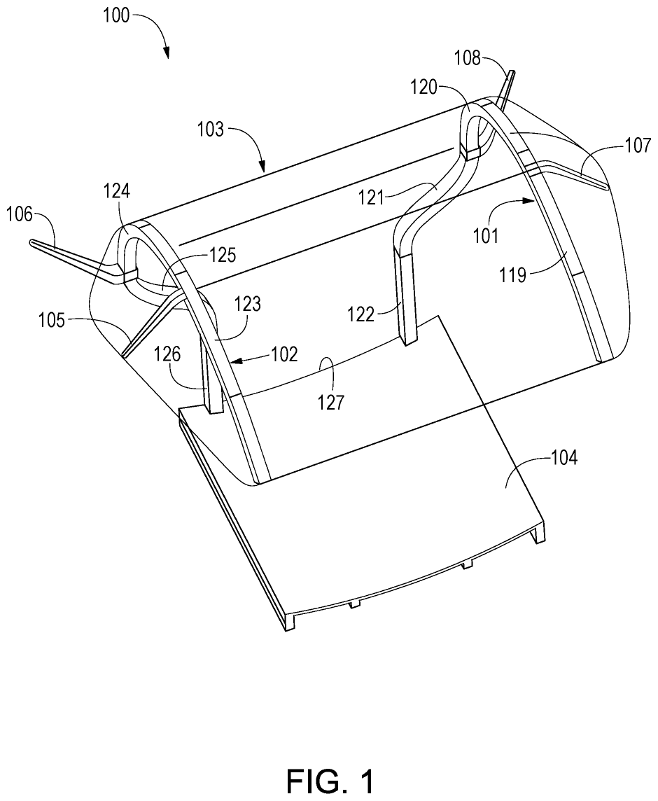

FIG. 1 is a perspective view of an upper body shield 100 in accordance with an embodiment of the present disclosure. The upper body shield 100 comprises a headrest 104 on which a patient lays his/her head when in a supine position.

Further, the upper body shield 100 comprises a right frame arm 101 and a left frame arm 102. Attached over and/or to the right and left frame arms 101, 102 is a transparent top cover 103. The transparent top cover 103 shields the patient partially on a back side of the upper body shield 100, on lateral sides of the upper body shield 100, and on a top side of the upper body shield 100. Thus, the patient's head (not shown) and the patient's chest (not shown) are partially covered by the transparent top cover 103.

Note that in one embodiment, the transparent top cover 103 is transparent plastic. However, the top cover 103 may be made of different types of materials in other embodiments. Notably, the transparent characteristic of the top cover 103 enables a medical professional to see the patient (not show).

The right frame arm 101 is configured to support the top cover 103. In this regard, the right frame arm 101 comprises a suspended arm 119 that extends with a radius of curvature downwardly. The suspended arm 119 suspends the top cover 103 over the patient's head and chest.

The right frame arm 101 further comprises a downward bend 120 integral with the suspended arm 119. The downward bend 120 provides spatial efficiency within the upper body shield 100.

Also, the right frame arm 101 comprises an s-shaped transition rod 121 that extends downward at a right angle, extends inwardly, and is integral with a vertical leg 122. The vertical leg 122 couples to a back edge 127 of the head rest 104. The vertical leg 122 may be coupled to the headrest 104, for example, by clips (not shown).

The left frame arm 102 is configured to support the top cover 103. In this regard, the left frame arm 102 comprises a suspended arm 123 that extends with a radius of curvature downwardly. The suspended arm 123 suspends the top cover 103 over the patient's head and chest.

The left frame arm 102 further comprises a downward bend 124 integral with the suspended arm 123. The downward bend 124 provides spatial efficiency within the upper body shield 100.

Also, the left frame arm 102 comprises an s-shaped transition rod 125 that extends downward at a right angle, extends inwardly, and is integral with a vertical leg 126. The vertical leg 126 couples to a back edge 127 of the head rest 104. The vertical leg 126 may be coupled to the headrest 104, for example, by clips (not shown).

The upper body shield 100 further comprises a right lateral support arm 107 that extends at an obtuse angle downwardly from the right frame arm 101. Additionally, the upper body shield 100 further comprises a left lateral support arm 105 that extends at an obtuse angle downwardly from the left frame arm 102. In use, the top cover 103 couples to and is supported by the right lateral support arm 107 and the left lateral support arm 105.

Note that the top cover 103 may be attached to the right frame arm 101 and left frame arm 102 in any number of ways. In addition to attaching to the right lateral support arm 107 and the left lateral support arm 104, the top cover 103 may attached to the right frame arm 101 and left frame arm 102 via epoxy, Velcro, or any other of attachment means that hold the top cover 103 in position to shield the head and chest of the patient from the medical professional.

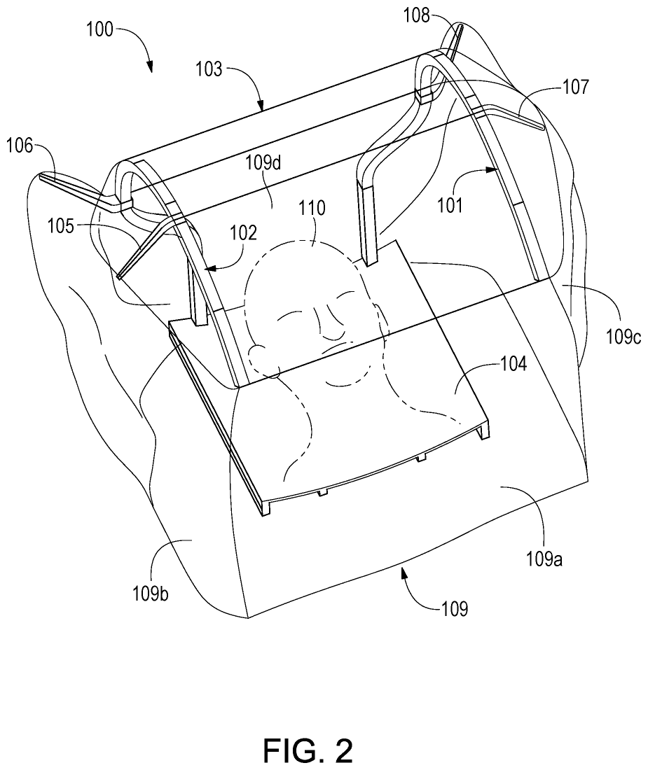

Further, the upper body shield 100 comprises a right dorsal support arm 108 and a left dorsal support arm 106. The right dorsal support arm 108 and a left dorsal support arm 106 support a drape (shown in FIG. 2) that provides cover for the front, sides, and back of the upper body shield 100.

FIG. 2 is a perspective view of the upper body shield 100 as shown in FIG. 1 with the addition of a drape 109 in accordance with an embodiment of the present disclosure. In this regard, the upper body shield 100 comprises the top cover 103 that is coupled to the right frame arm 101, the left frame arm 102, the right lateral support arm 107, and the left lateral support arm 105. Further, the right frame arm 101 and the left frame arm 102 are coupled to the headrest 104.

The drape 109 comprises a right side portion 109c that shields the right side of a patient 110. Also, the drape 109 comprises a front portion 109a that shields the head, neck, and chest of the patient 110. The drape 109 has as a left side portion 109b that shields the left side of the patient 110, and a back portion 109d that shields the head of the patient 110.

In one embodiment, the drape 109 is coupled to a right dorsal support arm 108 and a left dorsal support arm 106. The right dorsal support arm 108 and a left dorsal support arm 106 extend backward and outwardly providing spatial efficiency within the upper body shield 100.

In another embodiment, the drape 109 is coupled to the top cover 103. In this regard, the drape 109 may be coupled to the top cover 103 with Velcro, epoxy, clips, snaps, or the like.

In another embodiment, the drape 109 may be coupled at attachment points (not shown) along the right frame arm 101 and the left frame arm 102. The drape may be coupled to the right frame arm 101 and the left frame arm 102 via Velcro, epoxy, snaps, clips, or the like.

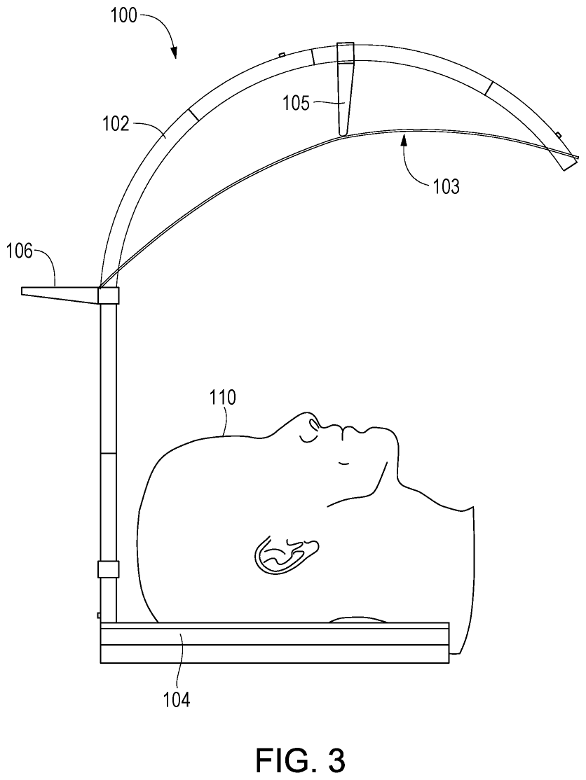

FIG. 3 is a side view of the upper body shield 100 without the drape 109 (FIG. 2) in accordance with an embodiment of the present disclosure. The upper body shield 100 comprises the headrest 104 on which a patient 110 rests his/her head.

The left frame arm 102 extends with a radius of curvature over the head of the patient 110. Further, the top cover 103, which is coupled to the left lateral support arm 105 may also be coupled to attachment points on the left frame arm 102. The left frame arm 102 couples to the headrest 104, e.g., via clips.

The upper body shield 100 comprises the dorsal support arm 106. The dorsal support arm 106 is configured to support the drape 109 (FIG. 2).

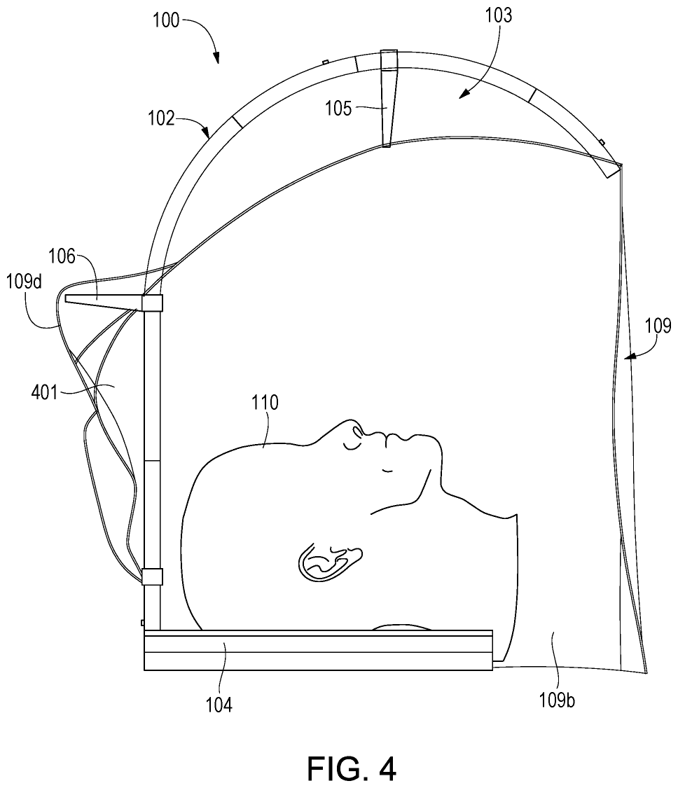

FIG. 4 is a side view of the upper body shield 100 with the side drape 109b and back drape 109d in accordance with an embodiment of the present disclosure. The upper body shield 100 comprises the headrest 104 on which a patient 110 rests his/her head.

The left frame arm 102 extends with a radius of curvature over the head of the patient 110. Further, the top cover 103, which is coupled to the left lateral support arm 105 may also be coupled to attachment points on the left frame arm 102. The left frame arm 102 couples to the headrest 104, e.g., via clips.

The upper body shield 100 comprises the dorsal support arm 106. The dorsal support arm 106 is configured to support the drape 109. In one embodiment, the back drape 109d is coupled to the dorsal support arm 106.

The dorsal support arm 106 extends backward and outwardly creating an opening 401 in the back drape 109d. In use, a medical professional (not shown) may insert his/her hand and/or arm through the opening 401. Thus, the opening 401 allows the medical professional to perform procedures on the patient 110.

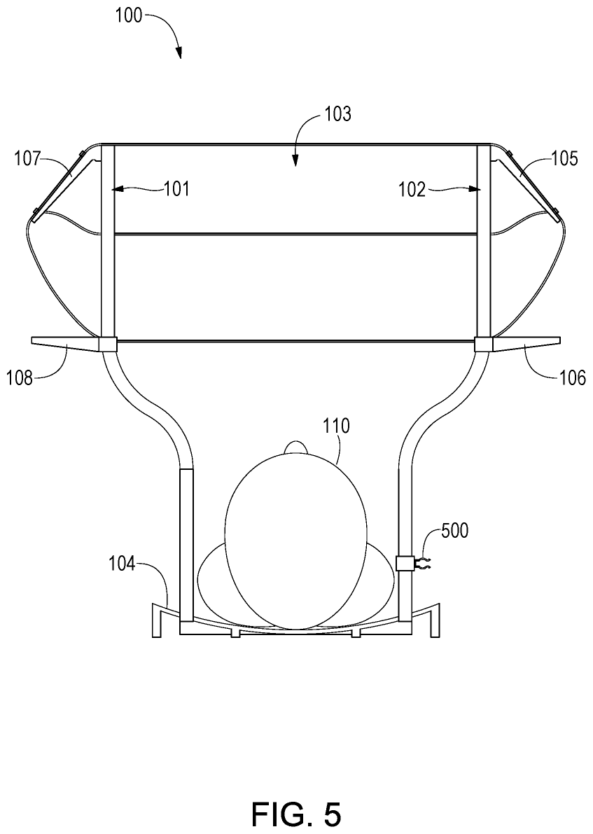

FIG. 5 is a back view of the upper body shield 100 without the drape 109 (FIG. 2) in accordance with an embodiment of the present disclosure. The head of the patient 110 rests on the headrest 104.

The right frame arm 101 extends with a radius of curvature over the head of the patient 110. The right frame arm 101 further extends downwardly and couples to the headrest 104.

The left frame arm 102 extends with a radius of curvature over the head of the patient 110. The left frame arm 102 further extends downwardly and couples to the headrest 104.

The top cover 103 extends over a portion of the right frame arm 101 and the left frame arm 102, creating a shield over the head of the patient 110. In one embodiment, the top cover is coupled to the right lateral support arm 107 and the left lateral support arm 105. The top cover 103 also be coupled to attachment points on the right frame arm 101 and the left frame arm 102. The right frame arm 101 and the left frame arm 102 couple to the headrest 104, e.g., via clips.

The upper body shield 100 comprises the dorsal support arm 106 and dorsal support arm 108. The dorsal support arm 106 and the dorsal support arm 108 are configured to support the drape 109 (FIG. 2), which is described further herein.

Note that in one embodiment, the upper body shield 100 may comprise an attachment clip 500. The retaining clip 500 may grasp a tube (not shown) that is being used with the patient 110. In one embodiment, the retaining clip 500 is moveable and may be moved to other structures or positions of the upper body shield 100.

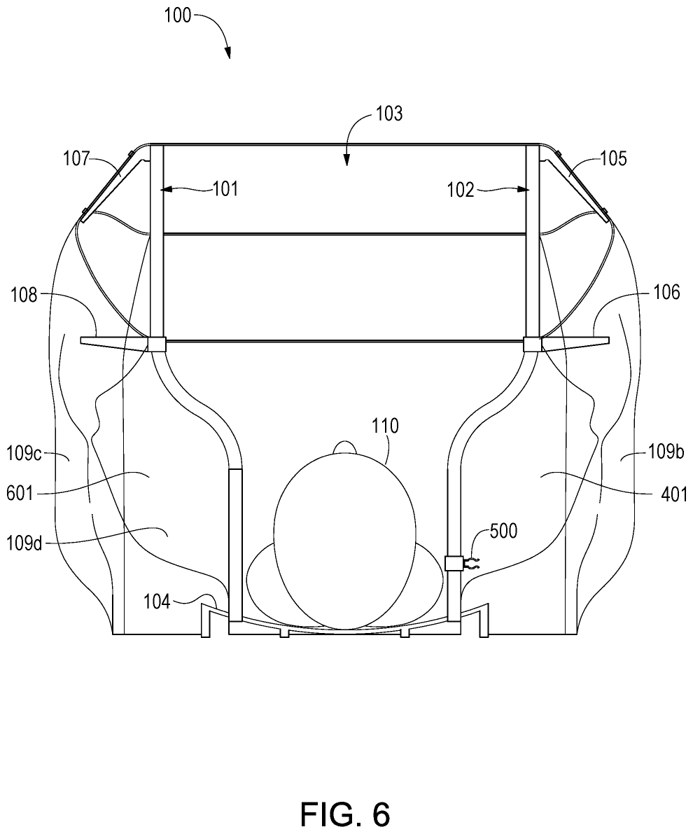

FIG. 6 is a back view of the upper body shield 100 with the drape 109 in accordance with an embodiment of the present disclosure. The head of the patient 110 rests on the headrest 104.

The right frame arm 101 extends with a radius of curvature over the head of the patient 110. The right frame arm 101 further extends downwardly and couples to the headrest 104.

The left frame arm 102 extends with a radius of curvature over the head of the patient 110. The left frame arm 102 further extends downwardly and couples to the headrest 104.

The top cover 103 extends over a portion of the right frame arm 101 and the left frame arm 102, creating a shield over the head of the patient 110. In one embodiment, the top cover is coupled to the right lateral support arm 107 and the left lateral support arm 105. The top cover 103 also be coupled to attachment points on the right frame arm 101 and the left frame arm 102. The right frame arm 101 and the left frame arm 102 couple to the headrest 104, e.g., via clips.

The upper body shield 100 comprises the dorsal support arm 106 and dorsal support arm 108. The dorsal support arm 106 and the dorsal support arm 108 are configured to support the drape 109. Note that the drape 109 comprises a front portion 109a, a right portion 109c, a left portion 109b, and a back portion 109d.

Note that in one embodiment, the upper body shield 100 may comprise an attachment clip 500. The attachment clip 500 may grasp a tube (not shown) that is being used with the patient 110. The attachment clip 500 may be configured for movement to another structure or position of the upper body shield 100.

In one embodiment, the back portion of the drape 109d of comprises openings 601, 401. A medical professional providing care to the patient 110 may extend his/her hands and arms through the openings 601, 401 to provide the care.

1. An upper body shield, comprising:
a headrest configured to balance and support the upper body shield;
at least one curved frame arm configured to attach to the headrest;
a top cover that couples to the at least one curved frame arm configured to shield a patient's head and neck;
a drape configured to couple to the at least one frame arm and configured to shield sides of the patient thereby shielding the patient from medical personnel and shielding the medical personnel from the patient during a procedure being performed by the medical professional.

2. The upper body shield of claim 1, wherein the at least one curved frame arm comprises a suspended arm that couples to the top cover over the patient's head and neck.

3. The upper body shield of claim 2, wherein the at least one curved frame arm comprises a downward bend integral with the suspended arm that provides spatial efficiency.

4. The upper body shield of claim 3, wherein the at least one curved frame arm comprises an s-shaped transition rod integral with the downward bend that extends downward at a right angle.

5. The upper body shield of claim 4, wherein the at least one curved frame arm comprises a vertical leg integral with the s-shaped transition rod.

6. The upper body shield of claim 5, wherein the vertical leg couples to a back edge of the head rest.

7. The upper body shield of claim 1, wherein the top cover is substantially transparent.

8. The upper body shield of claim 1, wherein the drape is substantially transparent.

9. The upper body shield of claim 1, wherein a back side of the drape comprises at least one opening for receiving a hand and/or arm.
